(19) United States
(12) Patent Application Publication
(10) Pub. No.: US 2026/0151129 A1
(43) Pub. Date: Jun. 4, 2026

(54) RETRACTION TORQUE MONITORING OF SURGICAL STAPLER

(71) Applicant: Covidien LP, Mansfield, MA (US)

(72) Inventors: Brock Kopp, Colorado Springs, CO (US); David N. Fowler, Cheshire, CT (US)

(21) Appl. No.: 18/706,828

(22) PCT Filed: Dec. 5, 2022
(86) PCT No.: PCT/IB2022/061782

§ 371 Date: May 2, 2024

Related U.S. Application Data
(60) Provisional application No. 63/286,722, filed on Dec. 7, 2021.

(51) Int. Cl. A61B 17/072; A61B 17/00; A61B 34/37; A61B 90/00

(52) U.S. Cl. CPC A61B 17/07207; A61B 34/37; A61B 90/06; A61B 2017/00119; A61B 2017/00199; A61B 2017/00398; A61B 2017/00477; A61B 2017/07278; A61B 2090/066

(57) ABSTRACT

An adapter for use with a surgical robotic system or a handheld powered surgical instrument couples a stapler loading unit to an instrument drive unit. The adapter may include a storage device storing a torque and/or current threshold that is used by a controller to limit torque imparted on the adapter during retraction of the stapler loading unit to prevent damage to the loading unit or the adapter.

### BACKGROUND

Surgical robotic systems may include a surgeon console controlling one or more surgical robotic arms, each having a surgical instrument having an end effector. In operation, the robotic arm is moved to a position over a patient and the surgical instrument is guided into a small incision via a surgical access port or a natural orifice of a patient to position the end effector at a work site within the patient's body. The surgical instrument may be a surgical stapler having an end effector configured to clamp, fasten, and cut tissue. A drive mechanism may be advanced to approximate a pair of jaws of the end effector while simultaneously ejecting fasteners and cutting tissue. After completing the procedure, the jaws of the surgical stapler are opened to release tissue by reversing the drive mechanism.

### SUMMARY

The present disclosure provides a stapling loading unit including a pair of jaw members, e.g., anvil and staple cartridge, that are closed and opened by advancing and retracting a drive mechanism actuated by one or more motors. During retraction, force imparted on the loading unit is monitored, to prevent damage to the loading unit and/or other components of the surgical instrument. The maximum force imparted on the loading unit may be limited by setting a maximum motor torque and/or current for one or more motors actuating the drive mechanism during retraction. This allows for surgical instruments and/or adapters to be manufactured to different tolerances with different torque thresholds that varies across various instruments/adapters.

The loading unit, which may be a single use or reusable unit, may be attached, either directly or using an adapter, to a robotic surgical system or a powered handheld surgical instrument. When present, the adapter may include a storage device configured to store a maximum retraction force, which may be provided as a maximum torque and/or current and/or force, that is used by the robotic surgical system or powered handheld surgical instrument to operate its motor(s) to limit forces experienced by the loading unit and/or adapter during retraction of the drive mechanism.

According to one embodiment of the present disclosure, a surgical robotic system is disclosed. The surgical robotic system includes a robotic arm having an instrument drive unit having one or more motors. The system also includes a loading unit having: a staple cartridge with a plurality of staples; an anvil to form the plurality of staples upon firing; and a drive beam configured to move at least one of the staple cartridge or the anvil relative to each other. The system further includes an adapter configured to couple to the instrument drive unit (or powered handle) having at least one motor configured to move the drive beam and a controller configured to limit a parameter of the at least one motor during retraction of the drive beam to prevent damage to at least one of the adapter or the loading unit.

Implementations of the above embodiment may include one or more of the following features. According to one aspect of the above embodiment, the surgical robotic system may include a sensor configured to measure the parameter of the at least one motor or a force imparted on the adapter. The sensor may be configured to measure at least one of torque or current draw. The controller may be further configured to compare the parameter to a threshold. The adapter may include a storage device configured to store the threshold and the controller may be configured to receive the threshold from the storage device. The controller may be further configured to stop the retraction of the drive beam in response to the parameter being equal to or above the threshold. The controller may be further configured to operate the at least one motor at a first speed during the retraction of the drive beam. The controller may be further configured to operate the at least one motor at a second speed, that is slower than the first speed, in response to the parameter being equal to or above the threshold. The controller may be further configured to output an alert that the retraction of the drive beam is stopped. The controller may be further configured to activate a manual mode enabling manual decoupling of the adapter from the instrument drive unit.

In further implementations, multiple (e.g., three or more) slower speeds corresponding to different torque or current thresholds before outputting a fault condition.

Another implementation may include a force sensor (e.g., strain gauge(s)) within the adapter configured to measure the retraction force. The robotic system may then compare retraction force to a threshold and control retraction based on the directly measured retraction force.

According to another embodiment of the present disclosure, a method for controlling a surgical stapler is disclosed. The method includes activating via a controller at least one motor of an instrument drive unit coupled to a loading unit including: a staple cartridge having a plurality of staples; an anvil to form the plurality of staples upon firing; and a drive beam configured to move at least one of the staple cartridge or the anvil relative to each other. The method also includes retracting the drive beam via the at least one motor. The method further includes limiting a parameter of the at least one motor during retraction of the drive beam to prevent damage to the loading unit.

Implementations of the above embodiment may include one or more of the following features. According to one aspect of the above embodiment, the method may include measuring the parameter of the at least one motor using a sensor. Measuring the parameter may include measuring at least one of torque or current draw. The method may also include comparing the parameter to a threshold at the controller. The method may further include receiving the threshold at the controller from a storage device configured to store the threshold. The method may additionally include stopping the retraction of the drive beam in response to the parameter being equal to or above the threshold. The method may also include operating the at least one motor at a first speed during the retraction of the drive beam. The method may further include operating the at least one motor at a second speed, that is slower than the first speed, in response to the parameter being equal to or above the threshold. The method may additionally include outputting an alert that the retraction of the drive beam is stopped. The method may further include activating a manual mode enabling manual decoupling of the adapter from the instrument drive unit.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a perspective view, with parts separated, of the instrument drive unit and a surgical instrument according to an embodiment of the present disclosure;

FIG. 6 is a perspective view of the surgical instrument of FIG. 5 in an unarticulated position;

FIG. 7 is an enlarged, perspective view of an end effector of the surgical instrument of FIG. 5 in an unarticulated position;

FIG. 8 is a flow chart of a method for controlling the surgical instrument of FIG. 5; and

FIG. 9 is a perspective view of a powered handheld surgical device including the surgical instrument of FIG. 5 according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “proximal” refers to the portion of the surgical robotic system and/or the surgical instrument coupled thereto that is closer to a base of a robot, while the term “distal” refers to the portion that is farther from the base of the robot.

As will be described in detail below, the present disclosure is directed to a surgical robotic system, which includes a surgeon console, a control tower, and one or more mobile carts having a surgical robotic arm coupled to a setup arm. The surgeon console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

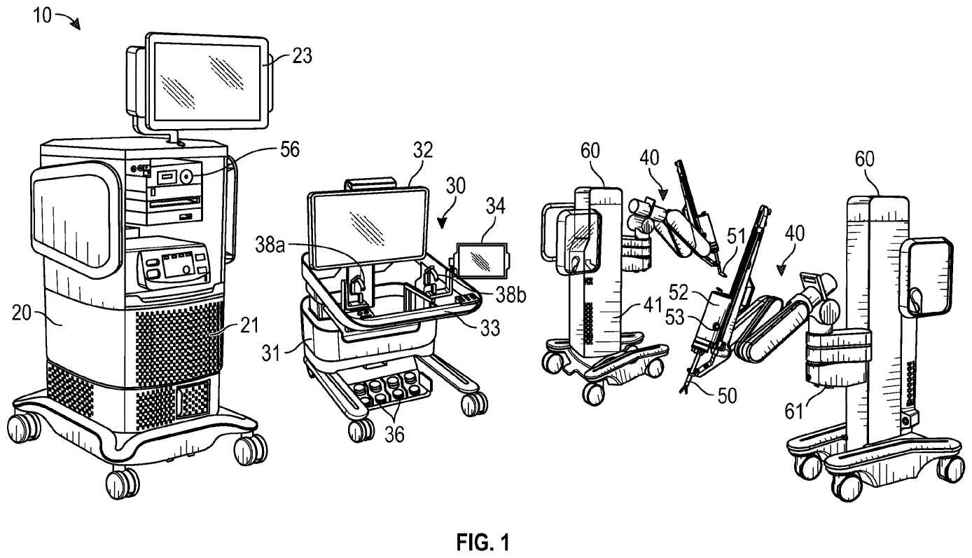

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 removably coupled thereto. The robotic arm 40 is also coupled to the movable cart 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the present disclosure and output the processed video stream.

The surgeon console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

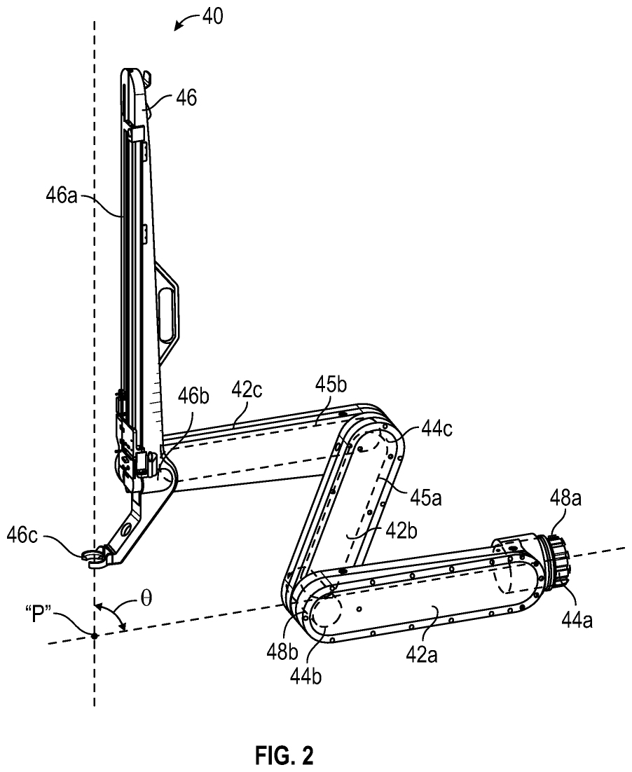

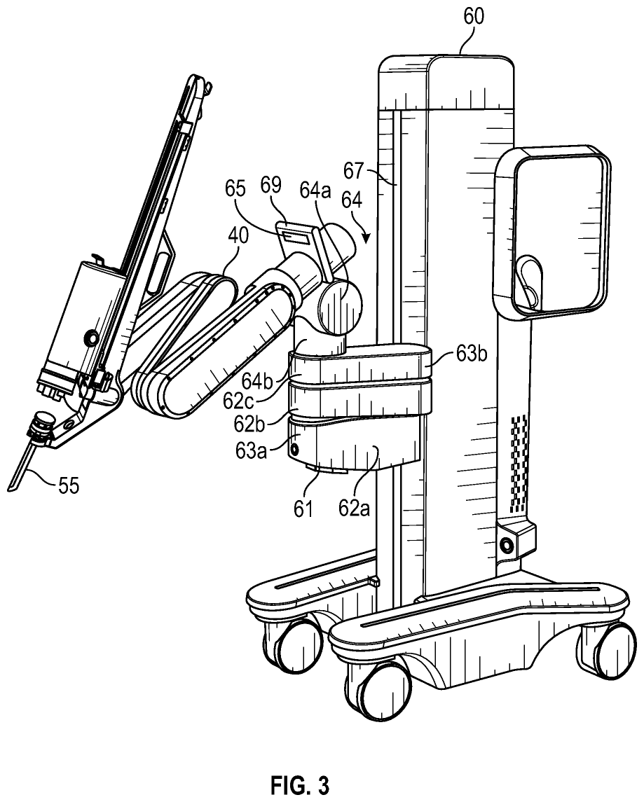

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48 b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ0. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and is configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46. The holder 46 also includes a port latch 46c for securing the port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

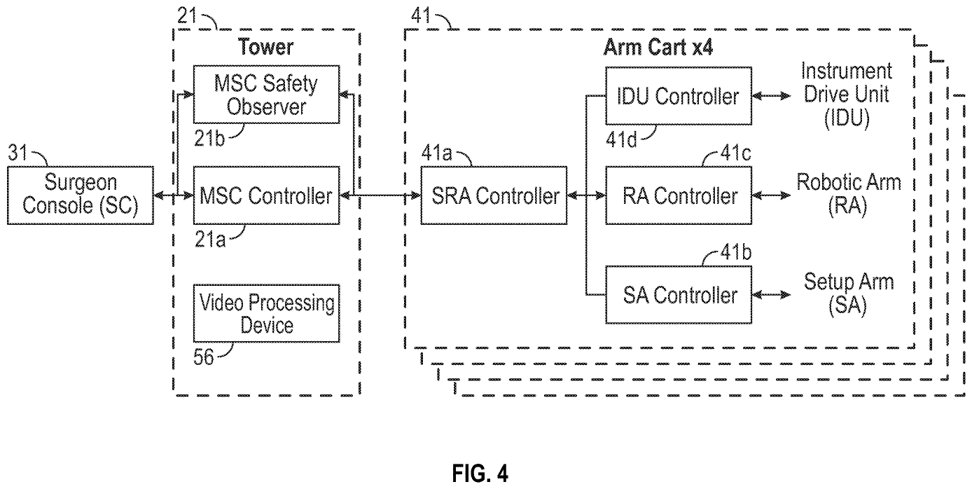

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (RPY) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

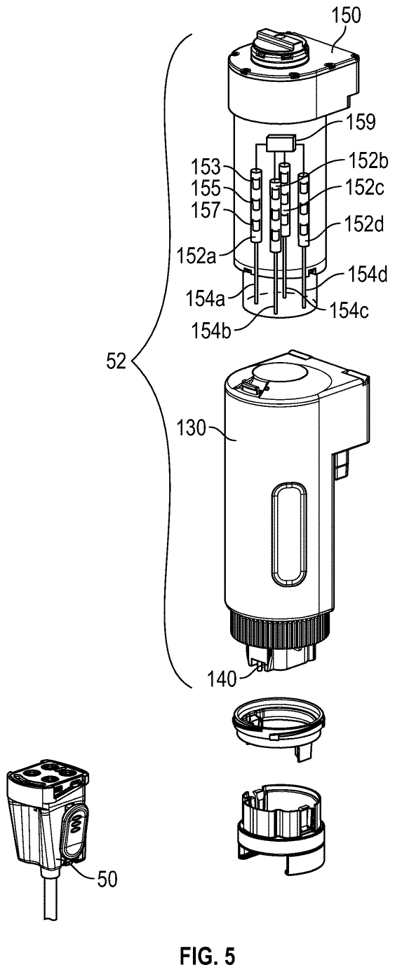

With reference to FIG. 5, the IDU 52 is shown in more detail and is configured to transfer power and actuation forces from its motors 152a, 152b, 152c, 152d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

The IDU 52 includes a motor pack 150 and a sterile barrier housing 130. Motor pack 150 includes motors 152a, 152b, 152c, 152d for controlling various operations of the instrument 50 The instrument 50 is removably couplable to IDU 52. As the motors 152a, 152b, 152c, 152d of the motor pack 150 are actuated, rotation of the drive transfer shafts 154a, 154b, 154c, 154d of the motors 152a, 152b, 152c, 152d, respectively, is transferred to the drive assemblies of the instrument 50.

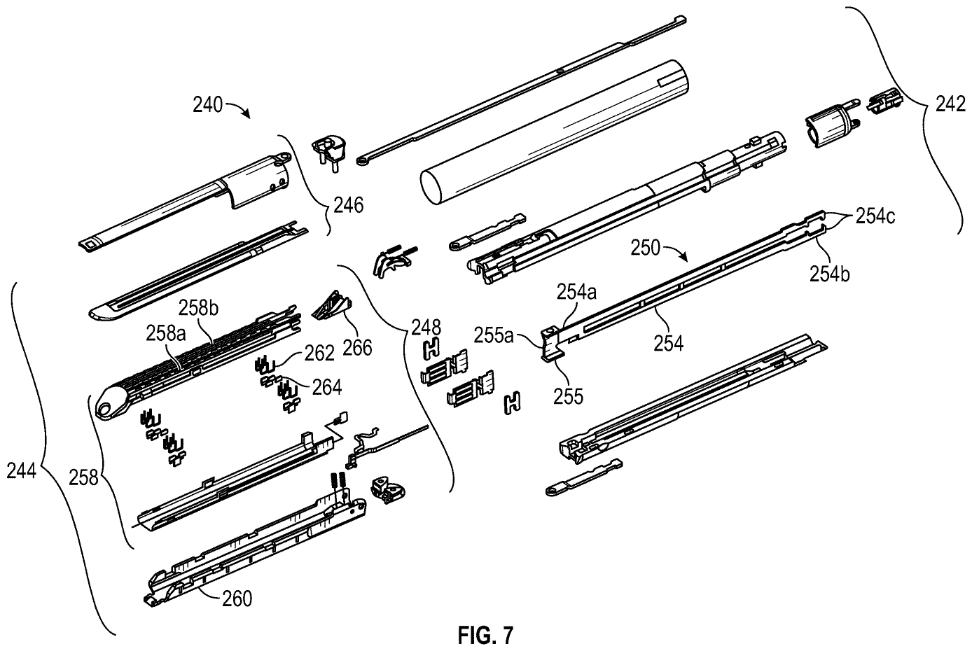

The instrument 50 is configured to couple to a loading unit 240 secured to a distal end thereof. The instrument 50 is configured to transfer rotational forces/movement supplied by the IDU 52 (e.g., via the motors 152a, 152b, 152c, 152d of the motor pack 150) into longitudinal movement or translation of the cables or drive shafts to effect various functions of an end effector 244 (FIG. 7).

Each of the motors 152a, 152b, 152c, 152d includes a current sensor 153, a torque sensor 155, and an encoder 157. For conciseness only operation of the motor 152a is described below. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by the motor 152a. The encoder 157 may be any device that provides a sensor signal indicative of the number of rotations of the motor 152a, such as a mechanical encoder or an optical encoder. Parameters which are measured and/or determined by the encoder 157 may include speed, distance, revolutions per minute, position, and the like. The sensor signals from sensors 153, 155, 157 are transmitted to the IDU controller 41d, which then controls the motors 152a, 152b, 152c, 152d based on the sensor signals. In particular, the motors 152a, 152b, 152c, 152d are controlled by an actuator controller 159, which controls torque outputted and angular velocity of the motors 152a, 152b, 152c, 152d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

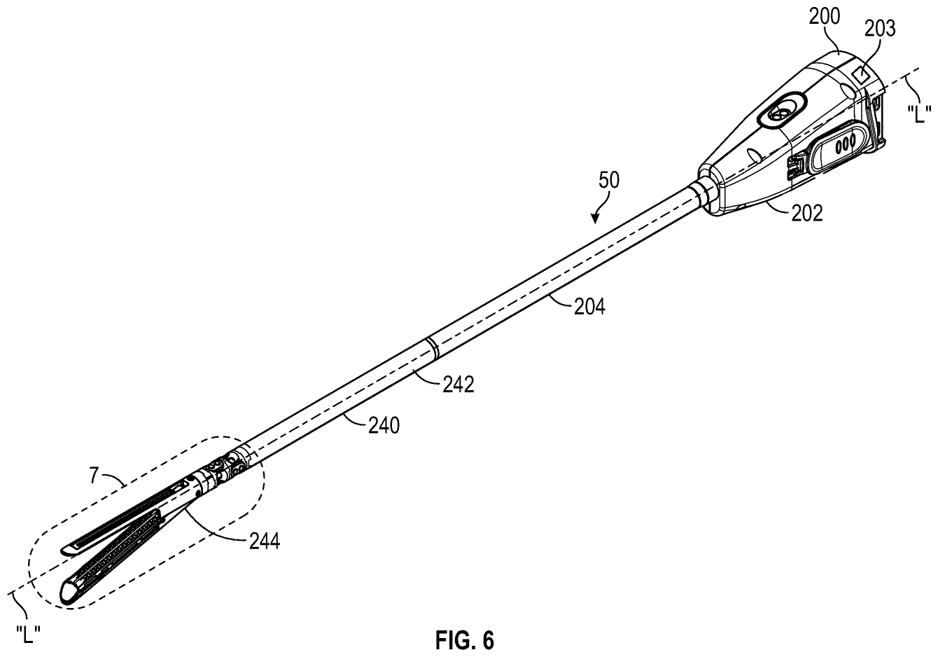

With reference to FIG. 6, instrument 50 includes an adapter 200 having a housing 202 at a proximal end portion thereof and an elongated shaft 204 that extends distally from housing 202. Housing 202 of adapter 200 is configured to selectively couple to IDU 52 of robotic surgical assembly 100, to enable motors 152a, 152b, 152c, 152d of IDU 52 of robotic surgical assembly 100 to operate the loading unit 240 coupled to the instrument 50. Housing 202 of adapter 200 supports a drive assembly that mechanically and/or electrically cooperates with motors 152a, 152b, 152c, 152d of IDU 52 of robotic surgical assembly 100. Drive assembly 250 of instrument 50 may include any suitable electrical and/or mechanical component to effectuate driving force/movement.

Elongated shaft 204 is configured to couple to the loading unit 240 having an end effector 244. With reference to FIGS. 6 and 7, the loading unit 240 includes a proximal body portion 242 and the end effector 244. Proximal body portion 242 is releasably attached to a distal end portion of the instrument 50, and end effector 244 is pivotally attached to a distal end of proximal body portion 242. End effector 244 includes an anvil assembly 246 and a cartridge assembly 248. Anvil assembly 246 is pivotable in relation to the cartridge assembly 248 and is movable between an open or unclamped position and a closed or clamped position. Proximal body portion 242 includes a drive assembly 250.

Drive assembly 250 includes a drive beam 254, which may be flexible, and having a distal end portion 254a and a proximal engagement section 254b. The distal end portion 254a includes an I-beam 255 having a knife 255 a. The I-beam 255 is configured to travel through the anvil assembly 246 and the cartridge assembly 248, thereby pushing the anvil assembly 246 toward the cartridge assembly 248 to clamp tissue. The proximal engagement section 254b includes diametrically opposed inwardly extending fingers 254c that engage a drive member (not shown) of the instrument 50 to fixedly secure drive member to the proximal end of flexible drive beam 254. Drive member is actuated by the IDU 52.

Cartridge assembly 248 of end effector 244 includes a staple cartridge 258 removably supported in a carrier 260. Staple cartridge 258 defines a central longitudinal slot 258a, and a plurality of linear rows of staple retention slots 258b positioned on each side of the central longitudinal slot 258a. Each of the staple retention slots 258b receives a staple 262 and a portion of a staple pusher 264. During operation, drive assembly 250 abuts an actuation sled 266 and pushes actuation sled 266 through the staple cartridge 258. As the actuation sled 266 moves through staple cartridge 258, cam wedges of the actuation sled 266 sequentially engage staple pushers 264 to move staple pushers 264 vertically within staple retention slots 258b and sequentially eject the staples 262 therefrom for formation against an anvil plate 246a of anvil assembly 246. In addition, the drive beam 254 closes the anvil assembly 246 and the cartridge assembly 248 and simultaneously advances the knife 255a and the actuation sled 266. Once clamping, cutting, and stapling is completed, the drive beam 254 is retracted in a reverse (i.e., proximal) direction.

The adapter 200 includes a storage device 203 configured to store various operating parameters pertaining to the adapter 200. Such parameters may include, for example, a maximum torque and/or maximum current that may be used during retraction of the drive beam 254 to open anvil assembly 246 and the cartridge assembly 248 to release stapled and cut tissue. The IDU controller 41d may obtain the parameters automatically by reading the parameters from the storage device 203 and/or the parameters may be set manually by the user by selecting either the type of the adapter 200 and/or the loading unit 240. The storage device 203 may be any suitable device configured to store data, e.g., flash memory.

The adapter 200 may also include a torque or force sensor (not shown) in addition or in lieu of the torque sensors 155 of the IDU 52.

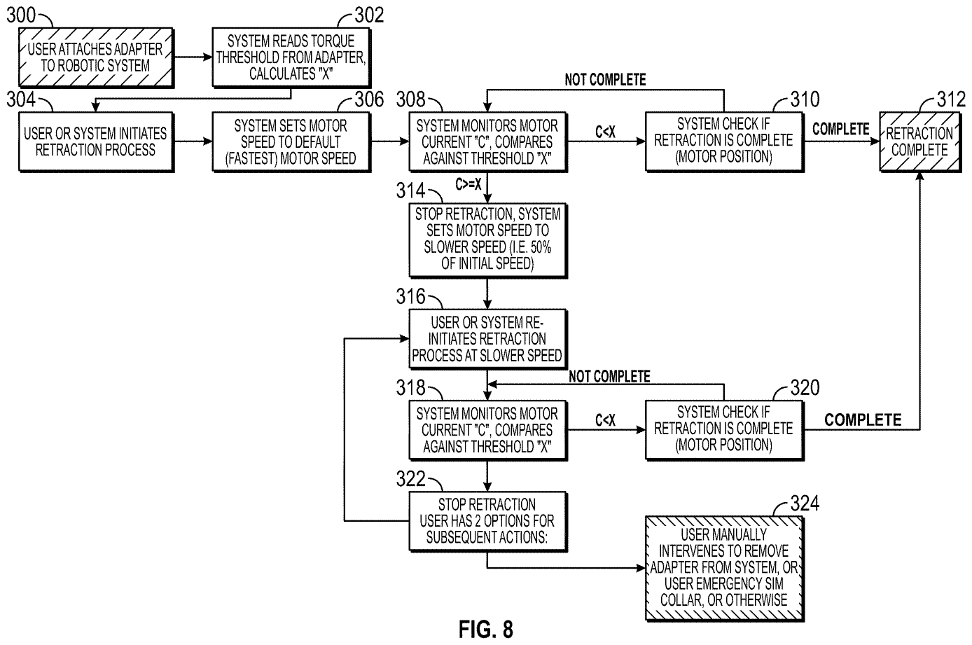

With reference to FIG. 8, at step 300 the adapter 200 is coupled to the IDU 52. At step 302, the IDU controller 41d accesses the data stored on the storage device 203 of the adapter 200, including a torque or current threshold for operating the motors 152a-d during retraction of the drive beam 254. The storage device 203 may be coupled to the IDU controller 41d using any wired or wireless communication interface. In embodiments, the IDU controller 41d may read one value, e.g., torque, and calculate a corresponding value, e.g., current. In embodiments, the torque or current thresholds may be stored in the system 10 and may be loaded based on the type of the adapter 200, which may be identified manually by the user or automatically by reading an identifier, e.g., from storage device 203, RFID, barcode, etc.

The torque and current thresholds may be identified for each adapter 200 individually or for any group of adapters 200 (e.g., model, batch, etc.). The thresholds may be established during manufacturing and testing the adapter 200 by calculating average retraction torque, maximum torque, a percentile of measured torque, etc.

Threshold torque may be also determined by measuring torque at two or more forces of different magnitude that are applied to the adapter 200 during manufacture/testing. A linear slope or rate of change between two torque values may be calculated to relate force and torque to each other. The resulting slope or rate of change may be expressed as one or more coefficients, which may be stored along with the threshold in the storage device 203. Similarly, multiple force inputs may be used to calculate a non-linear equation representing a relationship between torque and force and the coefficient and/or the equation may be written in the storage device 203. Thereafter, the IDU controller 41d may determine the threshold torque using the stored coefficients and/or equations.

At step 304, the IDU controller 41d initiates the retraction process. At step 306, the IDU controller 41d sets the speed of one or more of the motors 152a-d responsible for retracting the drive beam 254. The speed may be set to a default value, e.g., fastest speed. At step 308, the IDU controller 41d monitors current and/or torque via the current sensor 153 and/or the torque sensor 155. The IDU controller 41d compares the measured current and/or torque values to the threshold. At step 310, if the measured value is below the threshold, the IDU controller 41d verifies if the retraction is complete. This may be done by monitoring rotations of the motors 152a-d retracting the drive beam 254. Once retraction is verified, the IDU controller 41d notifies the system 10, which may output a message or as indication at step 312 that retraction is complete.

At step 314, if the measured value is at or above the threshold, the IDU controller 41d may command the motors 152a-d to stop the retraction process and may set the motor to a slower speed (e.g., about 50% of initial speed) (i.e., limit the speed parameter). In embodiments, retraction may be prevented, i.e., stopped without resuming. At step 316, the IDU controller 41d reinitiates the retraction process in response to a user command or an automated input from the system 10, e.g., in response to a first failed retraction attempt. In other embodiments, the retraction may continue at a different speed without stopping or subsequent user input.

During the second, i.e., slower, retraction process, at step 316, the IDU controller 41d monitors current and/or torque via the current sensor 153 and/or the torque sensor 155. At step 318, the IDU controller 41d compares the measured current and/or torque values to the threshold. At step 320, if the measured value is below the threshold, the IDU controller 41d verifies if the retraction is complete. Once retraction is verified, the IDU controller 41d notifies the system 10, which may output a message or an indication at step 312 that retraction is complete.

In embodiments, multiple (i.e., three or more) speeds may be used with corresponding torque or current thresholds until the retraction process is stopped. Similar logic decision may be used, such that each time a lower torque or current threshold is used, the retraction speed is adjusted to a different (i.e., slower) speed until retraction is successfully complete or stopped due to a mechanical fault.

At step 322, if the measured value is at or above the threshold, the IDU controller 41d commands the motors 152a-d to stop the retraction process. The system 10, at step 324, enters a manual mode allowing for the user to manually intervene by decoupling the adapter 200 from the IDU 52, which allows for manual manipulation of the end effector 244.

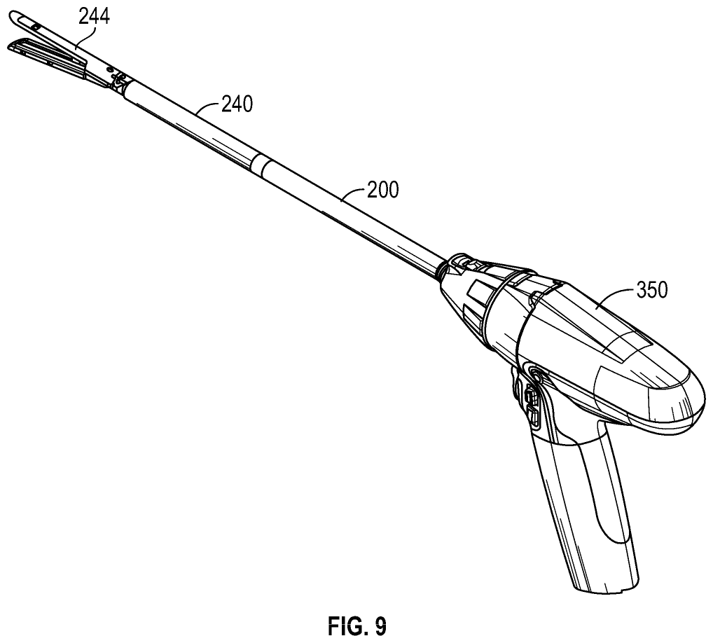

With reference to FIG. 9, the loading unit 240 may be used with a powered surgical handle 350, having similar components as the IDU 52, e.g., one or more motors, controllers, memory storing instructions, etc. The handle 350 is configured to control extraction in the similar manner described above.

It will be understood that various modifications may be made to the embodiments disclosed herein. In embodiments, the sensors may be disposed on any suitable portion of the robotic arm or stapling adapter. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a robotic arm including an instrument drive unit having at least one motor;
a loading unit including:
a staple cartridge having a plurality of staples;
an anvil to form the plurality of staples upon firing; and
a drive beam configured to move at least one of the staple cartridge or the anvil relative to each other;
an adapter configured to couple the loading unit to the instrument drive unit so the at least one motor can retract the drive beam; and
a controller configured to limit a parameter of the at least one motor during retraction of the drive beam to prevent damage to at least one of the adapter or the loading unit.

2. The surgical robotic system according to claim 1, further comprising a sensor configured to measure the parameter of the at least one motor or a force imparted on the adapter.

3. The surgical robotic system according to claim 2, wherein the sensor is configured to measure at least one of torque or current draw.

4. The surgical robotic system according to claim 1, wherein the controller is further configured to compare the parameter to a threshold.

5. The surgical robotic system according to claim 4, wherein adapter includes a storage device configured to store the threshold and the controller is configured to receive the threshold from the storage device.

6. The surgical robotic system according to claim 4, wherein the controller is further configured to stop the retraction of the drive beam in response to the parameter being equal to or above the threshold.

7. The surgical robotic system according to claim 6, wherein the controller is further configured to operate the at least one motor at a first speed during the retraction of the drive beam.

8. The surgical robotic system according to claim 7, wherein the controller is further configured to operate the at least one motor at a second speed, that is slower than the first speed, in response to the parameter being equal to or above the threshold.

9. The surgical robotic system according to claim 6, wherein the controller is further configured to output an alert that the retraction of the drive beam is stopped.

10. The surgical robotic system according to claim 6, wherein the controller is further configured to activate a manual mode enabling manual decoupling of the adapter from the instrument drive unit.

11. A method for controlling a surgical stapler, the method comprising:
activating, via a controller, at least one motor of an instrument drive unit coupled to a loading unit, the loading unit including:
a staple cartridge having a plurality of staples;
an anvil to form the plurality of staples upon firing; and
a drive beam configured to move at least one of the staple cartridge or the anvil relative to each other;
retracting the drive beam via the at least one motor; and
limiting a parameter of the at least one motor during retraction of the drive beam to prevent damage to the loading unit.

12. The method according to claim 11, further comprising:
measuring the parameter of the at least one motor using a sensor.

13. The method according to claim 12, wherein measuring the parameter includes measuring at least one of torque or current draw.

14. The method according to claim 11, further comprising:
comparing the parameter to a threshold at the controller.

15. The method according to claim 14, further comprising:
receiving the threshold at the controller from a storage device configured to store the threshold.

16. The method according to claim 14, further comprising:
stopping the retraction of the drive beam in response to the parameter being equal to or above the threshold.

17. The method according to claim 16, further comprising:
operating the at least one motor at a first speed during the retraction of the drive beam.

18. The method according to claim 17, further comprising:
operating the at least one motor at a second speed, that is slower than the first speed, in response to the parameter being equal to or above the threshold.

19. The method according to claim 16, further comprising:
outputting an alert that the retraction of the drive beam is stopped.

20. A surgical robotic system comprising:
a robotic arm including an instrument drive unit having at least one motor;
a loading unit including:
a staple cartridge having a plurality of staples;
an anvil to form the plurality of staples upon firing; and
a drive beam configured to move at least one of the staple cartridge or the anvil relative to each other;
an adapter configured to couple the loading unit to the instrument drive unit so the at least one motor can retract the drive beam; and
a controller configured to stop the at least one motor during retraction of the drive beam to prevent damage to at least one of the adapter or the loading unit in response to a measured motor parameter exceed a threshold.
